(19) United States
(12) Patent Application Publication
(10) Pub. No.: US 2006/0014957 A1
(43) Pub. Date: Jan. 19, 2006

(54) Resolution of an aryl-fused azapolycyclic compound

(75) Inventors: David C. Whritenour, Mystic, CT (US); Jason D. McKinley, Amston, CT (US); Ruth E. McDermott, Salem, CT (US); John A. Ragan, Gales Ferry, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/174,328

(22) Filed: Jul. 1, 2005

Related U.S. Application Data
(60) Provisional application No. 60/586,163, filed on Jul. 7, 2004.

(51) Int. Cl. C07D 221/22

(52) U.S. Cl. 546/97

(57) ABSTRACT

A process of obtaining an enantiomerically enriched salt from a racemic mixture of a compound of the formula 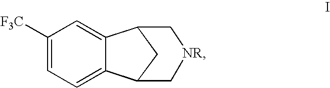 wherein R is hydrogen or benzyl.

### BACKGROUND OF THE INVENTION

The present invention relates to novel methods for the optical resolution of the racemic aryl-fused azapolycyclic compound 4-trifluoromethyl-10-aza-tricyclo[6.3.1.0^{2,7}]dodeca-2(7),3,5-triene having the formula

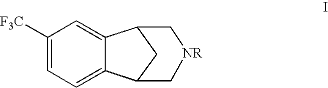

wherein R is H or benzyl, by formation of enantiomerically enriched salts of di-p-toluoyl-D-(−)-tartaric acid or di-p-toluoyl-L-(+)-tartaric acid and (+)-camphorsulfonic acid.

Aryl-fused azapolycyclic compounds, such as that of formula I bind to neuronal nicotinic acetylcholine specific receptor sites and are useful in modulating cholinergic function. Such compounds are useful in the treatment of inflammatory bowel disease (including but not limited to ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrythmias, gastric acid hypersecretion, ulcers, pheochromocytoma, progressive supranuclear palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, senile dementia of the Alzheimer's type (AD), Parkinson's disease (PD), attention deficit hyperactivity disorder (ADHD) and Tourette's Syndrome.

The compound of formula I may also be used in combination with an antidepressant such as, for example, a tricyclic antidepressant or a serotonin reuptake inhibiting antidepressant (SRI), in order to treat both the cognitive decline and depression associated with AD, PD, stroke, Huntington's chorea or traumatic brain injury (TBI); in combination with muscarinic agonists in order to stimulate both central muscarinic and nicotinic receptors for the treatment, for example, of ALS, cognitive dysfunction, age-related cognitive decline, AD, PD, stroke, Huntington's chorea and TBI; in combination with neurotrophic factors such as NGF in order to maximize cholinergic enhancement for the treatment, for example, of ALS, cognitive dysfunction, age-related cognitive decline, AD, PD stroke, Huntington's chorea and TBI; or in combination with agents that slow or arrest AD such as cognition enhancers, amyloid aggregation inhibitors, secretase inhibitors, tau kinase inhibitors, neuronal anti-inflammatory agents and estrogen-like therapy.

Other compounds that bind to neuronal nicotinic receptor sites are referred to in U.S. Pat. No. 6,020,335, issued on Feb. 1, 2000. The foregoing application is owned in common with the present application, and is incorporated herein by reference in its entirety.

### SUMMARY OF THE INVENTION

The present invention relates to a process for the optical resolution of a racemic mixture of the compound of formula I, wherein R is hydrogen, by formation of a di-p-toluoyl-tartaric acid salt having an enantiomeric excess of a compound having the absolute stereochemical configuration of the compound of formula

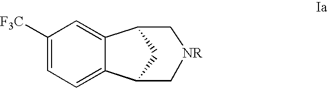

or an enantiomeric excess of a compound having the absolute stereochemical configuration of the compound of formula

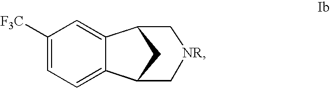

wherein (a) a molar amount of the racemic compound of formula I, wherein R is hydrogen, is dissolved in a reaction inert solvent, preferably acetonitrile or an alcoholic solvent selected from n-propanol, ethanol or methanol, (b) the solution of the racemic compound of formula I, wherein R is hydrogen, is treated with about an equimolar amount of di-p-toluoyl-D-(−)-tartaric acid or di-p-toluoyl-L-(+)-tartaric acid to form a mixture containing a suspended precipitate of di-p-toluoyl-tartaric acid salt, (c) the mixture containing the suspended precipitate is heated at about the boiling point of the solvent or below the boiling point of the solvent for about 0.5 hour to about 24 hours, with the precipitated material optionally checked for enantiomeric purity using standard means such as chiral HPLC and (d) the suspended precipitated enantiomerically enriched salt is collected by standard means, typically filtration. The enantiomerically enriched salt may then be treated with a base such as NaOH, KOH, Na₂CO₃, or K₂CO₃ and the like by standard means known in the art to liberate the enantiomerically enriched base.

The present invention also relates to a process for the optical resolution of a racemic mixture of the compound of formula I, wherein R is benzyl, by formation of the (+) camphorsulfonic acid salt having an excess of the compound of formula Ia.

### DETAILED DESCRIPTION OF THE INVENTION

The di-p-toluoyl-D-(−)-tartaric acid or di-p-toluoyl-L-(+)-tartaric acid salts of the compound of formula I, wherein R is hydrogen, enantiomerically enriched with the compound of formula Ia or formula Ib can be prepared according to the method exemplified by Scheme 1 which depicts treatment of a racemic mixture of the compound of formula Ia and formula Ib with di-p-toluoyl-D-(−)-tartaric acid and Scheme 2 which depicts treatment of said racemic mixture with di-p-toluoyl-L-(+)-tartaric acid. As will be evident to those skilled in the art, although the salts IIa and IIb in Scheme 1 and IIIa and IIIb in Scheme 2 and IVa in Scheme 3 are represented in neutral form, formulas IIa, IIb, IIIa, IIIb and IVa° C. also encompass charged forms in which there is proton transfer from the carboxylate to the amine function. Unless otherwise stated reaction conditions include an inert atmosphere commonly used in the art such as nitrogen or argon. The enantiomeric excess achieved by the process of the present invention ranges from about 65% to about 100%.

Scheme 1 refers to the preparation of the di-p-toluoyl-D-(−)-tartaric acid salt IIa having an enantiomeric excess of the compound with the absolute stereochemistry of formula Ia by dissolving a racemic mixture of Ia and Ib in a solvent selected from acetonitrile, n-propanol and ethanol, preferably acetonitrile, and then treating with about an equimolar amount of di-p-toluoyl-D-(−)-tartaric acid to form a mixture containing a suspended solid. The suspension is maintained with or without stirring at above about 35° C. to about the boiling point of the solvent, preferably about 40° C. to below the boiling point of the solvent, more preferably about 5° C. to about 10° C. below the boiling point of the solvent, preferably about 70° C. in the case of acetonitrile, for about 0.5 hours to about 26 hours, preferably about 1 hour to about 24 hours, during which time the optical purity of the suspended material may be optionally monitored by chiral HPLC. The suspension is cooled to about 35° C. and the enantiomerically enriched salt is separated, preferably by filtration.

Scheme 1 also refers to preparation of the di-p-toluoyl-D-(−)-tartaric acid salt IIb having an enantiomeric excess of the compound with the absolute stereochemistry of formula Ib by dissolving a racemic mixture of Ia and Ib in methanol and otherwise following the preceding process.

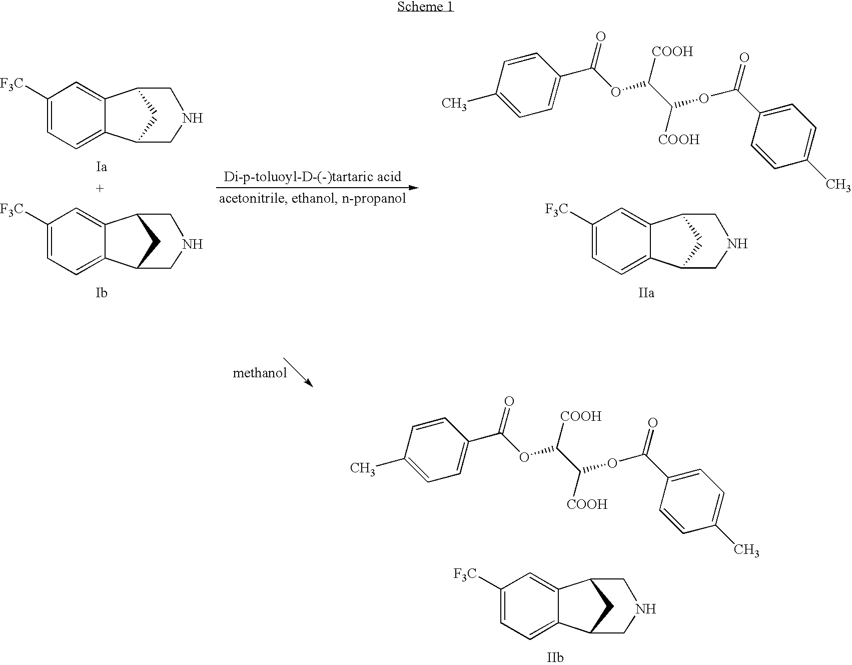

Scheme 2 refers to the preparation of the di-p-toluoyl-L-(+)-tartaric acid salt IIIb having an enantiomeric excess of the compound with the absolute stereochemistry of formula Ib by dissolving a racemic mixture of Ia and Ib in a solvent selected from acetonitrile, n-propanol and ethanol, preferably acetonitrile, and then treating with about an equimolar amount of di-p-toluoyl-L-(+)-tartaric acid to form a mixture containing a suspended solid. The suspension is maintained at above about 35° C. to about the boiling point of the solvent, preferably about 40° C. to below the boiling point of the solvent, more preferably about 5° C. to about 10° C. below the boiling point of the solvent, (preferably about 70° C. in the case of acetonitrile) for about 0.5 to about 26 hours, preferably about 1 hour to about 24 hours, during which time the optical purity of the suspended material may be optionally monitored by chiral HPLC. The suspension is brought to about 35° C. and the enantiomerically enriched salt is separated, preferably by filtration.

Scheme 2 also refers to preparation of the di-p-toluoyl-L-(+)-tartaric acid salt IIIa having an enantiomeric excess of the compound with the absolute stereochemistry of formula Ia by dissolving a racemic mixture of Ia and Ib in methanol and otherwise following the preceding process.

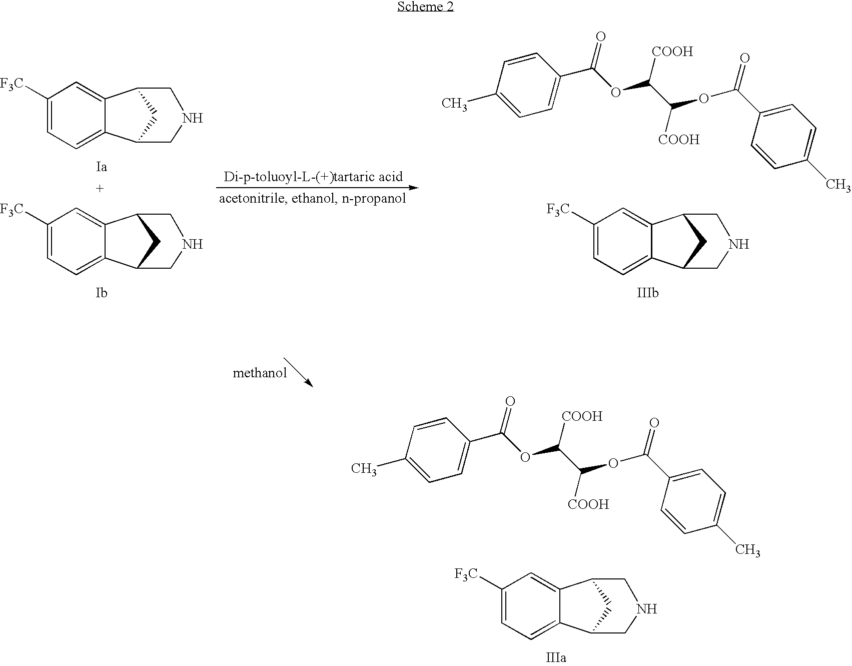

Scheme 3 refers to the preparation of the (+)-camphorsulfonic acid salt IVa of the compound of formula IV which is the compound of formula I, wherein R is benzyl, said salt having an enantiomeric excess of the compound with the absolute stereochemistry of formula

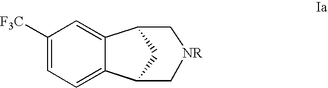

wherein R is benzyl.

The aforementioned salt is prepared by dissolving a racemic mixture of the compound of formula IV in a solvent selected from ethyl acetate, isopropyl ether and mixtures thereof, preferably mixtures thereof, most preferably a mixture of about equal volumes of ethyl acetate and isopropyl ether and then treating with about an equimolar amount of (+)-camphorsulfonic acid, preferably added as a solid, to form a mixture which is stirred at about room temperature or heated above room temperature, preferably at about 18° C. to about 30° C., more preferably at about 20° C. to about 25° C., for about 1 hour to about 24 hours, preferably about 12 hours to about 16 hours, and then collecting the resulting solids. The process of Scheme 3 produces an enantiomeric excess of about 65% to about 100% of the compound of formula IVa as the (+)-camphorsulfonic acid salt.

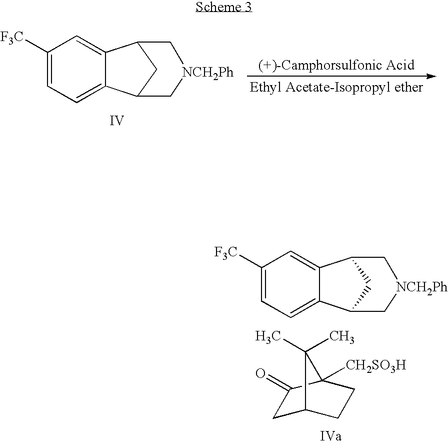

The following experimental examples illustrate, but do not limit the scope of, this invention.

### EXAMPLE 1

A racemic mixture of 4-trifluoromethyl-10-aza-tricyclo[6.3.1.0^{2,7}]dodeca-2(7),3,5-triene (1.0 g, 4.0 mmol, 1.0 equivalent) is dissolved in 10-20 ml of acetonitrile followed by addition of di-p-toluoyl-D-(−)-tartaric acid (1.8 g, 4.0 mmol, 1.0 equivalent) at 22° C. under N₂. The suspension that is formed is heated to 82° C. for 1 to 24 hours until enantiomeric purity is confirmed by chiral HPLC. The suspension is cooled to 35° C., filtered, and air dried for 1 hour. The crude (1S,8R)-(+)-4-trifluoromethyl-10-aza-tricyclo[6.3.1.0^{2,7}]dodeca-2(7),3,5-triene di-p-toluoyl-D-(−)-tartaric acid salt is then dried under vacuum at 40° C. for 24 hours to yield 1.3 grams of a white solid (2.1 mmol, 94% yield, 96% enantiomeric enrichment).

### EXAMPLE 2

The procedure of Example 1 was repeated using n-propanol as the solvent and heating the suspension to 83° C. A 95% enantiomeric enrichment with regard to (1S,8R)-(+)-4-trifluoromethyl-10-aza-tricyclo[6.3.1.0^{2,7}]dodeca-2(7),3,5-triene was achieved with a yield of 1.3 grams of a white solid (2.0 mmol, 90% yield)

### EXAMPLE 3

The procedure of Example 1 was repeated using ethanol as the solvent and heating the suspension to 79° C. An 80% enantiomeric enrichment with regard to (1S,8R)-(+)-4-trifluoromethyl-10-aza-tricyclo[6.3.1.0^{2,7}]dodeca-2(7),3,5-triene was achieved with a yield of 1.2 grams of a white solid (1.9 mmol, 84% yield)

### EXAMPLE 4

The procedure of Example 1 was repeated using methanol as the solvent and heating the suspension to 65° C. In contrast to the previous solvents a 72% enantiomeric enrichment with regard to the opposite enantiomer (1R,8S)-(−)-4-trifluoromethyl-10-aza-tricyclo[6.3.1.0^{2,7}]dodeca-2(7),3,5-triene was achieved with a yield of 1.1 grams of a white solid (1.7 mmol, 78% yield)

### EXAMPLE 5

The procedure of Example 1 was repeated using di-p-toluoyl-L-(+)-tartaric acid as the resolving agent. A 96% enantiomeric enrichment with regard to (1R,8S)-(−)-4-trifluoromethyl-10-aza-tricyclo[6.3.1.0^{2,7}]dodeca-2(7),3,5-triene was achieved with a yield of 1.3 grams of white solid (2.1 mmol, 93% yield)

### EXAMPLE 6

The procedure of Example 2 was repeated using di-p-toluoyl-L-(+)-tartaric acid as the resolving agent. A 95% enantiomeric enrichment with regard to (1R,8S)-(−)-4-trifluoromethyl-10-aza-tricyclo[6.3.1.0^{2,7}]dodeca-2(7),3,5-triene was achieved with a yield of 1.2 grams of a white solid (1.9 mmol, 88% yield)

### EXAMPLE 7

The procedure of Example 3 was repeated using di-p-toluoyl-L-(+)-tartaric acid as the resolving agent. An 80% enantiomeric enrichment with regard to (1R,8S)-(−)-4-trifluoromethyl-10-aza-tricyclo[6.3.1.0^{2,7}]dodeca-2(7),3,5-triene was achieved with a yield of 1.00 g of a white solid (1.6 mmol, 74% yield)

### EXAMPLE 8

The procedure of Example 4 was repeated using di-p-toluoyl-L-(+)-tartaric acid as the resolving agent. In contrast to the solvents of Examples 5-7, a 72% enantiomeric enrichment with regard to the opposite enantiomer (1S,8R)-(+)-4-trifluoromethyl-10-aza-tricyclo[6.3.1.0^{2,7}]dodeca-2(7),3,5-triene was achieved with a yield of 1.0 gram of a white solid (1.5 mmol, 70% yield)

### EXAMPLE 9

A racemic mixture of the N-benzyl derivative of 4-trifluoromethyl-10-aza-tricyclo[6.3.1.0^{2,7}]dodeca-2(7),3,5-triene (254 mg, 0.80 mmol) is dissolved in 5 volumes each of ethyl acetate and isopropyl ether (1.26 mL each). Solid (+)-camphorsulfonic acid (186 mg, 0.80 mmol) is then added in a single portion, and the solution is stirred at room temperature overnight. The resulting solids are collected by filtration and dried in a vacuum oven to provide 126 mg (57% ) of the camphorsulfonic acid salt. Chiral HPLC analysis showed a 94% enantiomeric enrichment of the (1S,8R) 97% of the (1R,8S) 3% of its enantiomer). The melting point (uncorrected) of the salt was 192.5-194.2° C.

What is claimed is:

1. A process of obtaining an enantiomerically enriched salt from a racemic mixture of a compound of the formula 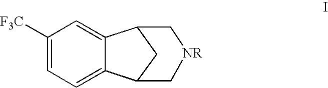
wherein R is hydrogen or benzyl, comprising the steps of (a) dissolving the racemic mixture of the compound of formula I in a solvent, (b) adding an approximately equimolar amount of an acid selected from di-p-toluoyl-D-(−)-tartaric acid, di-p-toluoyl-L-(+)-tartaric acid and (+)-camphorsulfonic acid to form a mixture containing a salt of compound I, (c) maintaining the mixture at about room temperature to about 50° C. below the boiling point of the solvent and (d) separating the enantiomerically enriched salt that forms from the mixture, with the proviso that (a) when R is hydrogen the acid selected is di-p-toluoyl-D-(−)-tartaric acid or di-p-toluoyl-L-(+)-tartaric acid and (b) when R is benzyl the acid selected is (+)-camphorsulfonic acid.

2. The process of claim 1 wherein R is hydrogen and the acid selected is di-p-toluoyl-D-(−)-tartaric acid or di-p-toluoyl-L-(+)-tartaric acid.

3. The process of claim 2 wherein the solvent is selected from acetonitrile, butyronitrile, proprionitrile, n-propanol, isopropanol, ethanol and methanol.

4. The process of claim 2 wherein the acid is di-p-toluoyl-D-(−)-tartaric acid.

5. The process of claim 2 wherein the acid is di-p-toluoyl-L-(+)-tartaric acid.

6. The process of claim 2 wherein the mixture is heated from about 40° C. to the boiling point of the solvent.

7. The process of claim 2 wherein the mixture is stirred.

8. The process of claim 2 wherein the solvent is selected from acetonitrile, n-propanol and ethanol, and about an equimolar amount of di-p-toluoyl-D-(−)-tartaric acid is added, and wherein the enantiomerically enriched salt comprises the di-p-toluoyl-D-(−)-tartaric acid salt having about a 65% to about a 100% enantiomeric excess of the compound of formula 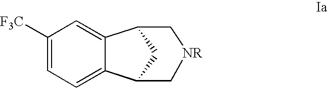
wherein R is hydrogen.

9. The process of claim 2 wherein the solvent is methanol and about an equimolar amount of di-p-toluoyl-D-(−)-tartaric acid is added, and wherein the enantiomerically enriched salt comprises the di-p-toluoyl-D-(−)-tartaric acid salt having about a 65% to about a 100% enantiomeric excess of the compound of formula 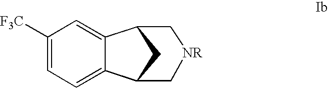
wherein R is hydrogen.

10. The process of claim 2 wherein the solvent is selected from acetonitrile, n-propanol or ethanol and about an equimolar amount of di-p-toluoyl-L-(+)-tartaric acid is added, and wherein the enantiomerically enriched salt comprises the di-p-toluoyl-L-(+)-tartaric acid salt having about a 65% to about a 100% enantiomeric excess of the compound of formula 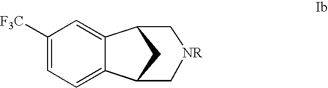
wherein R is hydrogen.

11. A di-p-toluoyl-D-(−)-tartaric acid or di-p-toluoyl-L-(+)-tartaric acid salt made by the process of claim 2 having an enantiomeric excess of a compound of formula 1a: 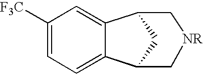
wherein R is hydrogen or an enantiomeric excess of the compound of formula Ib: 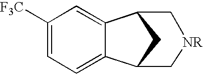
wherein R is hydrogen.

12. The L- or D-tartrate salt of 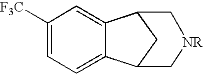
where R is H or benzyl, or a solvate thereof.

13. The di-p-toluoyl-L-(+)-tartaric acid or di-p-toluoyl-D-(−)-tartaric acid salt of 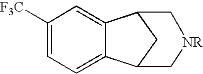
where R is H or benzyl, or a solvate thereof.

14. The (+) or (−)-camphorsulfonic acid salt of 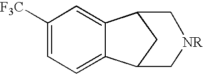
where R is H or benzyl, or a solvate thereof.
